(19) United States
(12) Patent Application Publication
(10) Pub. No.: US 2024/0009302 A1
(43) Pub. Date: Jan. 11, 2024

(54) Herpes 2 Antibody System

(71) Applicant: Raymond Joel Rairie, Manchester, NY (US)

(72) Inventor: Raymond Joel Rairie, Manchester, NY (US)

(21) Appl. No.: 18/220,256

(22) Filed: Jul. 10, 2023

Related U.S. Application Data
(63) Continuation-in-part of application No. 17/063,715, filed on Oct. 5, 2020, now abandoned.
Application No. 17/063,715 is a continuation-in-part of application No. 15/663,786, filed on Jul. 30, 2017, now abandoned.

(51) Int. Cl. A61K 39/245

(52) U.S. Cl. CPC A61K 39/245; A61K 2039/525

(57) ABSTRACT

The HERPES 2 ANTIBODY SYSTEM prevents people from getting HERPES 2. It prevents people from getting HERPES SIMPLEX 2 VIRUS (HSV-2) (HERPES 2) on their genitals, mouth, anus, hands, eyes and face. And it prevents a baby from getting infected with HERPES 2 during birth.

This application is a Continuation-In-Part of application Ser. No. 17/063,706, filed on Oct. 5, 2020, which was a Continuations-In-Part of application Ser. No. 15/663,786, filed on Jul. 30, 2017, both of which are incorporated by reference.

### FIELD

This application is designed to: (1) protect people from getting infected with HERPES SIMPLEX 2 (HSV-2) commonly known as “HERPES 2”, on or around their genitals, mouth, and anus, and (2) prevent babies from getting infected with HERPES 2 during birth (the baby could die if the baby gets infected). The HERPES 3 ANTIBODY SYSTEM involves the creation of a composition of matter, the matter being antibodies for HERPES 2.

### BACKGROUND

The purpose of this patent is to benefit humanity and society and civilization. People enjoy sex very much. Unfortunately—sometimes people contract Herpes on their private parts or mouth. Millions of people have Herpes on their private parts and mouth. Some people have a natural ability to fight off Herpes—this is good for them. Others are able to take medication to control outbreaks. Unfortunately, Herpes medication does not always work—or only works to a certain point. Unfortunately, some people have painful outbreaks on their private areas and mouth two or three or more times per year—for all their life. They lose work. And (unfortunately) sometimes (unintentionally) spread Herpes to their spouse or girlfriend or boyfriend. Herpes can (unfortunately) cause emotional pain. Also, it is very sad to say that Herpes can hurt or kill a baby—sometimes when a mother is giving birth—and the mother has Herpes—the baby can contract. Herpes during the birth. Because the baby is so young—the baby cannot fight off the infection. I would like to fix these sad issues.

### SUMMARY

This system will intentionally infect a person with Herpes Simplex 2. The infection shall be controlled and regulated. My system will also generate antibodies for HSV-2 (HERPES 2). My system will prevent people from getting HERPES 2 on their genitals and other sensitive areas, and on their mouths or other areas of the body. My system will also prevent babies from getting infected with HERPES 2 during birth.

The HERPES 2 ANTIBODY SYSTEM (THE SYSTEM) is a unique, complicated, time consuming, and expensive process that generates matter (HERPES 2 antibodies). The HERPES 2 ANTIBODY SYSTEM will not help people who are already infected with HERPES 2. If someone is only infected with HERPES 1 then they could benefit from THE SYSTEM—they could be protected from contracting HERPES 2. THE HERPES 1 AND 2 ANTIBODY SYSTEMS are the only SYSTEMS on the planet Earth that do these things. THE HERPES 2 ANTIBODY SYSTEM will benefit humanity, society, and civilization. It will prevent millions of people, over thousands of years from getting terrible and painful sores on their private areas. And it will save babies.

### BRIEF DESCRIPTION OF THE DRAWING

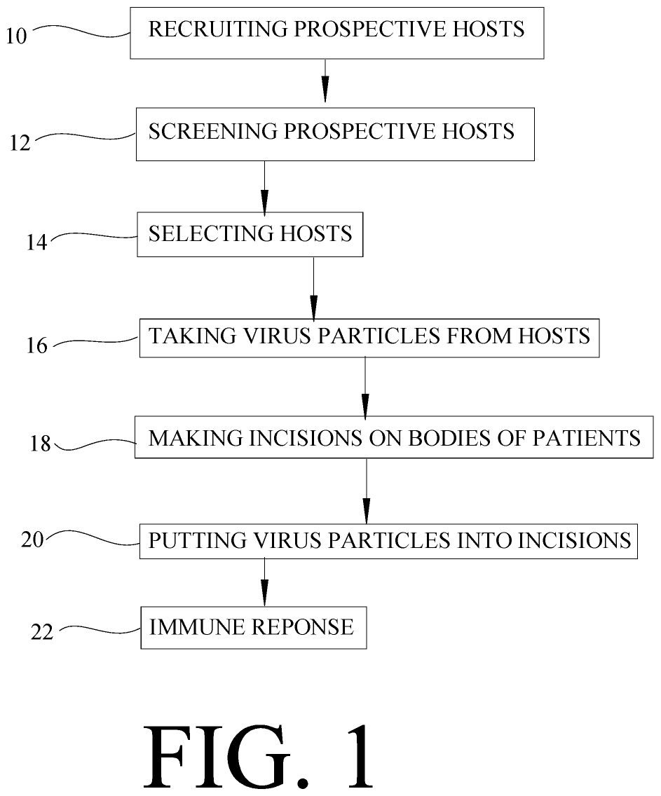

The sole drawing is FIG. 1, which is a flowchart illustrating the system of the invention.

### DESCRIPTION

THE HERPES 2 ANTIBODY SYSTEM (hereafter referred to as “THE SYSTEM”) is a highly regulated and controlled process. THE SYSTEM is designed to protect the PATIENT (described below) from the disease of HERPES SIMPLEX 2 HERPES 2. And also to protect a baby during birth from getting HERPES. THE SYSTEM is unique.

### The Medical Clinic

THE SYSTEM shall consist of a professional medical clinic (hereafter referred to as “the clinic”). The clinic shall be under the direct supervision of one or more licensed Medical Doctor(s). The doctor(s) shall have both training and experience in dealing with the disease of HERPES SIMPLEX 2 (HERPES 2). The clinic will also employ a professional HUMAN RESOURCE worker—to handle recruitment, direct control, and supervision of the HOST—(the HOST will be described below).

The medical clinic shall have the capacity to take blood samples from the HOSTS and the PATIENTS. A licensed nurse(s) shall obtain blood samples from the HOST and the PATIENT (the PATIENT shall be described below).

### The Patient

There will be two types of people who will utilize the clinic. One type of person will be the person who is currently not infected with HERPES 2. This type of person will be known as “The PATIENT”. The PATIENT is paying the clinic to provide the PATIENT with the HERPES 2 ANTI-BODY SYSTEM. The other person who will utilize the clinic will be a person who is already infected with HERPES 2. This already infected person shall be known as “the HOST”. The HOST is paid to provide HERPES 2 to the clinic. The HOST will be subjected to extensive screening of their past behavior, and must pass a physical exam, and psychological exam, and health tests (as described below). The HOST is financially compensated for their time and effort. The HOST is treated with great respect by THE SYSTEM. Finding the correct HOST is a key part. THE SYSTEM can be compared to finding the correct machine in order for a factory to work properly (like a cog in the machinery). Finding the proper HOST could be very difficult and expensive. The HOST must have a certain background in order for THE SYSTEM to work correctly. Everything must be put together just right—or THE HERPES 2 ANTIBODY SYSTEM will not work correctly. The PATIENT will be intentionally infected with HERPES 2. The intentional HERPES infection shall be regulated and controlled (as described below). Licensed medical doctor(s) and licensed nurses shall handle and supervise the process.

The Host Selecting the correct HOST is a key part to the HERPES 2 ANTIBODY SYSTEM. The HOST will be providing the clinic with HERPES 2 samples. The HOST will be tested for HERPES 1 and HERPES 2, and the AIDS virus. The HOST will be tested for the Hepatitis B virus. The HOST will be tested for the Hepatitis C virus. The HOST will be tested for Syphilis. The licensed nurse shall take the blood samples of the HOST at the clinic. The blood samples shall be subject to the latest, most reliable, and most sophisticated testing possible. The HUMAN RESOURCE worker will recruit the HOST. The HOST will be subject to questions regarding their personal behavior—such as illegal drug use and sexual activity. The HOST will be subject to psychological testing, screening, and background checks. The HOST shall be treated with the greatest respect in a way the host will be a VERY IMPORTANT PERSON (V.I.P.). A good comparison could be having the correct quarterback on a football team—or else it does not matter if you have other good players on the team. The reason the HOST is subject to these tests and procedures—is that the HOST will be providing the HERPES samples from visible HERPES sores on the HOST'S body. The transfer shall take place with a swab (as described below). The HOST must be in the proper physical condition and proper psychological condition to provide the PATIENT with Hepatitis B, nor Hepatitis C, nor Syphilis. The HOST will be subjected to psychological testing and screening. THE SYSTEM will not work correctly if the HOST does things—such as: injecting heroin the night before they are scheduled to contribute a HERPES sample and then exposing the PATIENT to blood-born infections. This is why it is so important to have the professional HUMAN RESOURCE worker as part of the SYSTEM. This part will take considerable time, effort, and money. The HUMAN RESOURCE worker will be responsible to keep the HOST from doing the wrong things.

The HUMAN RESOURCE worker and the DOCTOR(S) will analyze all relevant data to insure a potential HOST has all of the necessary factors to safely provide the clinic with HERPES HERPES 2. If this section of THE SYSTEM is not done correctly a patient could become infected with AIDS or HEPATITIS B or HEPATITIS C or Syphilis. The best and most sophisticated and most reliable medical tests will be used to determine if the HOST is infected with HERPES=I and/or HERPES 2 and if they have a current HERPES 1 or HERPES 2 outbreak on their lips.

When a person (the PATIENT) wants to gain protection from HERPES 2 infecting their genitals, mouth, anus, hands, face, and eyes—they can make an appointment with the clinic. At the clinic they will be subject to blood tests. The blood tests will be the best, most sophisticated, and most reliable possible. It will be determined if they are already infected with HERPES I or HERPES 2. If they are already infected with HERPES I they will not need THE SYSTEM for HERPES 1. But they could still use THE SYSTEM to get protected from HERPES 2. If they are already infected with HERPES 2 they will not need THE SYSTEM for HERPES 2. But they could still use THE SYSTEM to get protected from HERPES 1.

Once a person is infected with HERPES 2—that person will develop HERPES 2 antibodies. Once a person has HERPES 2 antibodies, they can no longer get HERPES 2. (But they could still have outbreaks at the original HERPES 2 infection area).

If the PATIENT is not infected with HERPES 1 nor infected with HERPES 2: then THE HERPES ANTIBODY SYSTEM will prevent the PATIENT from getting HERPES 1 and HERPES 2 on their genitals. And prevent the PATIENT from getting both HERPES 1 and HERPES 2 on or in their mouth. And prevent the PATIENT from getting both HERPES 1 and HERPES 2 on and inside their anus, also protecting their hands, face, and eyes.

If the PATIENT is already infected with HERPES 1—then THE SYSTEM will not protect them from HERPES 1. But if the PATIENT is not infected with HERPES 2—then THE SYSTEM will protect them from HERPES 2.

If the PATIENT is already infected with HERPES 2—then THE SYSTEM will not protect them from HERPES 2. But if the PATIENT is not infected with HERPES I—then THE SYSTEM will protect them from HERPES 1.

Blood tests at the clinic will determine if the PATIENT has already been infected with HERPES 1 and/or HERPES 2. If the PATIENT has already been with both HERPES 1 and HERPES 2 then THE SYSTEM cannot help them. If after the original blood tests it is determined that a PATIENT can benefit from THE SYSTEM: then the PATIENT is subject to the next step in the process.

The PATIENT will be informed of the next step in the SYSTEM. The Patient will be intentionally with: HERPES Il only; or HERPES 2 only; or both HERPES 1 and HERPES 2. The PATIENT will decide on what type of infection they will get after discussing the situation with the doctor(s).

The intentional infection process will take place at a location or locations on the PATIENT'S body that will not be as sensitive as their genitals, nor mouth, nor anus (which are normal areas in HERPES infections).

### The Swabbing

The intentional HERPES infection shall be at a location on the PATIENT'S body the PATIENT has selected to be intentionally infected. The PATIENT shall discuss the situation with the doctor(s) And then make a decision. If the PATIENT is only going to get one intentional HERPES infection, then only one location will be selected. But if the PATIENT is getting two intentional HERPES infections—then the PATIENT shall select two areas on their body to be infected. The PATIENT shall have full authority on where the intentional HERPES infection shall be. If the PATIENT is intentionally getting infected with both HERPES 1 and HERPES 2, the PATIENT will make the decision for both locations. Possible non-sensitive locations could be under the upper arm—by the armpit or on the top of a foot. Or some other area of the body—as determined by the PATIENT (after discussing the situation with the doctor(s)).

The area(s) of intentional HERPES infection shall be subjected to a medically professional incision made by the doctor(s) at the clinic. The equipment used for the incision shall be medically professional and medically sterile. The intentional infection shall be done by the doctor(s) or licensed nurse—using a swab. The doctor(s) shall determine the best material possible to make and use for the swab. The swab shall be medically professionally made and medically professionally disinfected and medically professionally clean—and kept in a medically professional sterile package. And kept in the medically professional sterile package until ready for immediate use.

The doctor(s) or licensed nurse shall take the sterile swab and use the sterile swab to make contact with the HOST'S visible HERPES 1 sore(s) on the HOST'S lips. And then intentionally transfer the HERPES I virus from the HOST'S lips to the PATIENT'S intentional incision for the HERPES I intentional infection.

The doctor(s) or licensed nurse shall take the sterile swab and use the sterile swab to make contact with the HOST'S visible HERPES 2 sore(s) on the HOST'S lips. And then intentionally transfer the HERPES 2 virus from the HOST'S lips to the PATIENT'S intentional incision for the HERPES 2 intentional infection.

If the PATIENT is only getting infected with HERPES I—then only one incision will be made. If the PATIENT is only getting infected with HERPES 2—then only one incision will be made. If the PATIENT is getting infected with both HERPES 1 and HERPES 2—then the doctor(s) will make two incisions. This is done to see if and when an original HERPES outbreak has occurred. One location will be for HERPES I and the other location will be for HERPES 2. The doctor(s) will keep a record of where each and what type of intentional infection took place. This will help the doctor(s) determine if an infection actually took place (as described below) or whether the intentional infection process (the swabbing process) needs to be repeated. And if it needs to be repeated, the doctor(s) will know which infection needs to be HERPES 1 or HERPES 2. Also, sophisticated, expensive, and reliable blood tests will be done about 21 to 42 days after the original outbreak both HERPES 1 and HERPES 2 to determine if HERPES antibodies have been generated by the PATIENT'S body. And exactly which HERPES antibodies have been generated (HERPES I or HERPES 2 or both).

### Infection

A typical HERPES 2 infection will occur from 2 days to 21 days after original infection. After 21 days the doctor(s) will conduct the most reliable, most modern, expensive, and sophisticated blood test(s) to determine if any and what type of HERPES infection(s) took place. If the PATIENT does not have a visible HERPES outbreak after 21 days—then it is possible that the PATIENT will not have a visible outbreak. But, the patient could still have been infected (meaning the intentional swabbing was effective). The blood tests will provide the doctors with the information needed to determine whether or not there was a HERPES 1 infection or a HERPES 2 infection or no infection at all. The doctor(s) will then wait another 21 to 42 days to conduct new sophisticated, most reliable, most modern, and expensive blood tests to determine whether or not HERPES 2 antibodies have been generated by the PATIENT.

If HERPES 2 antibodies have been detected, THE SYSTEM worked for HERPES 2. If HERPES 2 antibodies have not been detected then the swabbing did not generate a HERPES 2 infection. The process is repeated.

Once the PATIENT has antibodies for HERPES 2, then the PATIENT is immunized to getting a HERPES 2 infection anywhere else on their body. Once antibodies for HERPES 2 are generated by a person's body, the antibodies are generated for the life of that person.

### Protection for a Baby

If a mother has generated antibodies for both HERPES 1 and HERPES 2 with the HERPES ANTIBODY SYSTEM, then the mother does not need to worry about getting HERPES 1 nor HERPES 2, and there is no chance of her baby getting HERPES during birth.

### Flowchart for the Pinball Herpes 2 Antibody System (Shown in FIG. 1)

### ESTABLISH MEDICAL CLINIC and RECRUIT THE HOST 10

### CONDUCT BLOOD TESTS and PSYCHOLOGICAL SCREENING ON HOST 12

### SELECTING HOSTS 14

### GET PATIENT—START PROCESS

### CONDUCT BLOOD TESTS ON PATIENT

### PATIENT DECIDES WHAT ANTIBODIES DO

THEY WANT (HERPES 1 or HERPES 2 or BOTH)

### PATIENT DECIDES WHERE TO BE

INFECTED

### INTENTIONALLY INFECT PATIENT 16, 18, 20

### WAIT 2 to 21 DAYS for INFECTION TO GENERATE (OUTBREAK)

### BLOOD TESTS TO DETERMINE IF and

WHAT TYPE OF INFECTION TOOK PLACE

### WAIT 21 to 42 DAYS AFTER OUTBREAK

### TEST PATIENT FOR ANTIBODIES

### PATIENT IMMUNIZED or REPEAT PROCESS 22

STEP A: The Pinball Herpes Antibody System (Pinball System) shall prevent a person from contracting painful Herpes 1 (Herpes Simplex Virus 01) sores and prevent painful Herpes 2 sores (Herpes Simplex Virus 02) from infecting a person's genital area, and prevent infection in the anal area, and prevent infection in the mouth-face area. The Pinball System will also prevent a baby getting Neonatal Herpes (by preventing the baby from getting Herpes during birth—by ensuring the mother does not have genital Herpes). The Pinball System also prevents Encephalitis Herpes (by preventing Herpes from entering the brain).

STEP B: The Pinball System provides a Host (a healthy person)—who will already be infected with Herpes 1 and or Herpes 2. The Host will be medically screen to ensure they are not infected with Aids, Hepatitis B, Hepatitis C, Syphilis, or other communicable blood transferred pathogens. This is to protect the Patient (the person getting the Herpes protection) from getting any other infection. The Patient will be getting infected with Herpes 1 and/or Herpes 2—but the infection will be superficial and will not generate a Herpes infection at a sensitive area (genitals, anus, mouth) of the body. The Herpes infection will be intentional (but regulated and controlled).

STEP C: The Pinball System will utilize a professional and licensed medical doctor and nurse, in a professional and medically licensed clinic to infect the Patient with Herpes 1 and/or Herpes 2. The Doctor will obtain the proper amount of Herpes 1 and/or Herpes 2 from the Host (who will have an active Herpes outbreak)—this is known as the Virus Particles. The Virus Particles could also be stored in a medically professional storage system designed to store Herpes 1 and/or Herpes 2 Virus Particles. The amount of Herpes 1 and/or Herpes 2 Virus Particles will be greater than 10 to the 4th power—in order to reach the necessary threshold to ensure Herpes infection. This will induce said Patient's body to generate Herpes 1 and/or Herpes 2 antibodies. Said Doctor shall make a professional medical incision on said Patients intentional infection location (about half and inch incision). All precautions shall be done to ensure said Patient is not infected with any other pathogen. The medical procedure shall be conducted in a manner to ensure the Patients medical safety and comfort.

STEP D: The specific area of the intentional Herpes infection shall be at the Patient's upper inside back of the right leg—or the upper inside back of the left leg. This is known as the Infection Location. It is possible to use another Infection Location that the Patient prefers to be infected at. The specific Infection Location shall be connected, by nerves, to said Patient's Sacral Ganglia. When the Herpes 1 and/or Herpes 2 goes dormant—said Herpes will go dormant in said Patients Sacral Ganglia. This is a known medical fact. There may be a visible Herpes 1 and/or Herpes 2 sore at said Infection Location. But, said Herpes infection shall be minor—because of the limited amount of Herpes Particles that will be used, and the small area of infection, also said Infection Location has less sensitive nerves than the genital, anus, and mouth areas. This is a known medical fact.

STEP E: The Herpes 1 and/or Herpes 2 will infect the Patient at the Infection Location. The genitals, anus, and mouth will not be infected, and will therefore not have painful Herpes sores or Painful Herpes blisters.

STEP F: After initial infection at the Infection Location—said patient will develop Herpes antibodies to protect said Patient from becoming infected with Herpes at another area of said

Patients body. This is a known medical fact. It will take said Patient's body 21 to 42 days to develop said Herpes 1 and/or Herpes 2 antibodies. Once Herpes antibodies are generated—said Patient cannot be infected with Herpes 1 and/or Herpes 2 at a new area. This is a known medical fact. So as a result—said Patient is protected from getting infected with Herpes 1/and or Herpes 2 on their genitals, anus, and or mouth. Said Patient is now protected from any new Herpes infection.

STEP G: The Herpes virus will go dormant in the Sacral Ganglia because the Infection Location is connected to the Sacral Ganglia by nerves. This is a known medical fact. So the Herpes will not go dormant in said Patient's Trigeminal Ganglia (which is in the face area). So the Herpes will not transfer into said Patient's brain. So said Patient will not contract Herpes Encephalitis—the Herpes will not transfer into the brain from the Trigeminal Ganglia—because said Herpes virus will not be in the Trigeminal Ganglia—the Herpes 1 and/or Herpes 2 will be in the Sacral Ganglia (which is in the lower part of the body). This is a known medical fact.

STEP H: Because the Herpes is in the lower part of said Patients body (the regulated-controlled Infection Location)—said Patient will not get Herpes 1 and/or Herpes 2 on or in their lips or mouth. So said Patient is now protected from getting painful Herpes sores on the mouth. Once said Patient has Herpes antibodies their body has the capacity to protect all other areas of said Patients body from a new Herpes infection. This is a known medical fact.

STEP I: The Pinball Herpes Antibody System (Pinball System) prevents said Patient from contracting Herpes 1 and/or Herpes 2 from infecting sensitive areas of a person's body: the genitals, anus, and mouth area are protected from getting infected with Herpes. The only area of said Patient that contracts Herpes is the preselected upper inside back of the leg (or some other area determine by said Patient). The infection is intentional—but controlled and regulated. The actual infection is superficial—because of the small amount of Herpes entering said Patient. Almost all standard Herpes infections take place over three to seven minutes—the standard amount of time for intimate contact (which cause a standard Herpes 1 and/or Herpes 2 infection).

STEP J: The Pinball System manipulates the body to generate Herpes 1 and Herpes 2 antibodies. Once said Herpes antibodies are generated said Patient is protected from getting any Herpes infection. The said Patient is protected from getting Herpes 1 and/or Herpes 2—because the Pinball System has generated matter—said matter is Herpes Simplex Virus One (HSV-01) antibodies, and said matter is Herpes Simplex Virus Two (HSV-02) antibodies.

STEP K: The Pinball Herpes Antibody System (Pinball System) generates matter: HSV-01 antibodies and HSV-02 antibodies.

CONCLUSION: The Pinball System prevents people from getting Herpes 1 and/or Herpes 2 on their genitals, anus, and mouth. The Pinball System prevents Neonatal Herpes. The Pinball System prevents Encephalitis Herpes. The Pinball System generates matter.

1. A system for producing an immune response to Herpes-2 in humans, comprising the steps of:
recruiting prospective hosts, who are otherwise healthy persons who have already been infected with Herpes-2;
screening the prospective hosts for communicable blood transferred pathogens;
selecting hosts from those who have passed the screening;
taking virus particles from the hosts;
making incisions on intentional infection locations on the bodies of patients;
putting enough virus particles into the incisions to cause an immune response, but not so many as to produce painful sores.

2. The system for producing an immune response to Herpes-2 in humans according to claim 1, wherein:
the communicable blood transferred pathogens that are screened for include those that cause AIDS, Hepatitis, and syphilis.

3. The system for producing an immune response to Herpes-2 in humans according to claim 1, wherein:
the number of virus particles put into each incision shall be greater than ten thousand.

4. The system for producing an immune response to Herpes-2 in humans according to claim 1, wherein:
the intentional infection locations shall be on the upper inside back of one of each patient's leg.

5. The system for producing an immune response to Herpes-2 in humans according to claim 1, wherein:
the incisions are made by trained medical personnel.

6. The system for producing an immune response to Herpes-2 in humans according to claim 1, wherein:
after the virus particles are taken from the hosts,
they are placed in a medically professional storage system,
before they are placed into the incisions in the patients.

7. The system for producing an immune response to Herpes—in humans according to claim 1, wherein:
the system generates matter, namely, Herpes Simplex Virus 2 antibodies.

8. A system for producing immunity to Herpes-2 in humans, comprising the steps of:
recruiting prospective hosts, who are otherwise healthy persons who have already been infected with Herpes-2;
screening the prospective hosts for communicable blood transferred pathogens;
selecting hosts from those who have passed the screening;
taking virus particles from the hosts;
making incisions on intentional infection locations on the bodies of patients;
putting enough virus particles into the incisions to cause a sufficient immune response to create immunity to Herpes-2 in the patients.

9. The system for producing immunity to Herpes-2 in humans according to claim 8, wherein:
the communicable blood transferred pathogens that are screened for include those that cause AIDS, Hepatitis, and syphilis.

10. The system for producing immunity to Herpes-2 in humans according to claim 8, wherein:
the number of virus particles put into each incision shall be greater than ten thousand.

11. The system for producing immunity to Herpes-2 in humans according to claim 8, wherein:
the intentional infection locations shall be on the upper inside back of one of each patient's leg.

12. The system for producing immunity to Herpes-2 in humans according to claim 8, wherein:
the incisions are made by trained medical personnel.

13. The system for producing immunity to Herpes-2 in humans according to claim 8, wherein:
after the virus particles are taken from the hosts,
they are placed in a medically professional storage system,
before they are placed into the incisions in the patients.

14. The system for producing immunity to Herpes-2 in humans according to claim 8, wherein:
the system generates matter, namely, Herpes Simplex Virus 1 antibodies.

15. A method for producing a composition of matter, namely, antibodies to Herpes-2, comprising the steps of:
recruiting prospective hosts, who are otherwise healthy persons who have already been infected with Herpes-2;
screening the prospective hosts for communicable blood transferred pathogens;
selecting hosts from those who have passed the screening;
taking virus particles from the hosts;
making incisions on intentional infection locations on the bodies of patients;
putting enough virus particles into the incisions to cause a sufficient immune response to create immunity to Herpes-2 in the patients; and
the system generates matter, namely, Herpes Simplex Virus 1 antibodies.

16. The system for producing immunity to Herpes-2 in humans according to claim 15, wherein:
the communicable blood transferred pathogens that are screened for include those that cause AIDS, Hepatitis, and syphilis.

17. The system for producing immunity to Herpes-2 in humans according to claim 15, wherein:
the number of virus particles put into each incision shall be greater than ten thousand.

18. The system for producing immunity to Herpes-2 in humans according to claim 15, wherein:
the intentional infection locations shall be on the upper inside back of one of each patient's leg.

19. The system for producing immunity to Herpes-2 in humans according to claim 15, wherein:
the incisions are made by trained medical personnel.

20. The system for producing immunity to Herpes-2 in humans according to claim 15, wherein:
after the virus particles are taken from the hosts,
they are placed in a medically professional storage system,
before they are placed into the incisions in the patients.
